(19) United States
(12) Patent Application Publication
(10) Pub. No.: US 2007/0282373 A1
(43) Pub. Date: Dec. 6, 2007

(54) Hemostatic Pressure Plug

(76) Inventors: Mark Ashby, Laguna Niguel, CA (US); Roy D. Bertolet, Ormond Beach, FL (US); Andrew H. Cragg, Edina, MN (US); Tin Trong Tran, Anaheim, CA (US)

Correspondence Address: MILLER, MATTHIAS & HULL, ONE NORTH FRANKLIN STREET, SUITE 2350, CHICAGO, IL, 60606, US

(21) Appl. No.: 10/595,977

(22) PCT Filed: Nov. 24, 2004
(86) PCT No.: PCT/US04/39588

§ 371 Date: Jun. 14, 2007

Related U.S. Application Data
(60) Provisional application No. 60/525,355, filed on Nov. 25, 2003.

(51) Int. Cl. A61B 17/08

(52) U.S. Cl. 606/213

(57) ABSTRACT

An apparatus to intervascularly promote hemostasis at a blood vessel puncture site with an inner lumen pressure and an outer lumen pressure has a flexible plug having a center, a top surface, and a bottom surface, and a release mechanism coupled to the center to position and release the flexible plug intervascularly at the blood vessel puncture site. The inner lumen pressure is greater than the outer lumen pressure to forceably secure the flexible plug around the blood vessel puncture site.

### FIELD OF THE INVENTION

The invention relates to facilitating hemostasis at a puncture site. More particularly, the invention relates to facilitating hemostasis at a puncture site by utilizing the pressure difference between the inside and the outside of the blood vessel. Even more particularly, the invention relates to facilitating hemostasis at a puncture site by deploying a hemostatic plug within the blood vessel and utilizing the pressure difference between the inside and the outside of the blood vessel to secure the hemostatic plug around the puncture site.

### BACKGROUND OF THE INVENTION

A large number of diagnostic and interventional procedures involve the percutaneous introduction of instrumentation into a vein or artery. For example, coronary angioplasty, angiography, atherectomy, stenting of arteries, and many other procedures often involve accessing the vasculature through a catheter placed in the femoral artery or other blood vessel. Once the procedure is completed and the catheter or other instrumentation is removed, bleeding from the punctured artery must be controlled.

Traditionally, external pressure is applied to the skin entry site to stem bleeding from a puncture wound in a blood vessel. Pressure is continued until hemostasis has occurred at the puncture site. In some instances, pressure must be applied for up to an hour or more during which time the patient is uncomfortably immobilized. In addition, a risk of hematoma exists since bleeding from the vessel may continue beneath the skin until sufficient clotting effects hemostasis. Further, external pressure to close the vascular puncture site works best when the vessel is close to the skin surface but may be unsuitable for patients with substantial amounts of subcutaneous adipose tissue since the skin surface may be a considerable distance from the vascular puncture site.

There are several approaches to close the vascular puncture site including the use of anchor and plug systems as well as suture systems. The use of an anchor and plug system addresses these problems to some extent but provides other problems including: 1) complex and difficult application; 2) partial occlusion of the blood vessel by the anchor when placed properly; and 3) complete blockage of the blood vessel or a branch of the blood vessel by the anchor if placed improperly. Another problem with the anchor and plug system involves re-access. Re-access of a particular blood vessel site sealed with an anchor and plug system is not possible until the anchor has been completely absorbed because the anchor could be dislodged into the blood stream by an attempt to re-access the site.

Internal suturing of the blood vessel puncture requires a specially designed suturing device. These suturing devices involve a significant number of steps to perform suturing and require substantial expertise. Additionally, when releasing hemostasis material at the puncture site and withdrawing other devices out of the tissue tract, the user typically must pull or tug on the devices which may reposition the hemostasis material or cause damage to the surrounding tissue or vascular puncture site. Moreover, approaches to sealing the puncture utilizing suture systems only partially occlude the blood vessel puncture thereby allowing blood to seep out of the puncture thereby causing hematoma.

### BRIEF DESCRIPTION OF THE INVENTION

An apparatus to intervascularly promote hemostasis at a blood vessel puncture site with an inner lumen pressure and an outer lumen pressure has a flexible plug having a center, a top surface, and a bottom surface, and a release mechanism coupled to the center to position and release the flexible plug intervascularly at the blood vessel puncture site. The inner lumen pressure is greater than the outer lumen pressure to forceably secure the flexible plug around the blood vessel puncture site.

### BRIEF DESCRIPTION OF THE DRAWINGS

The accompanying drawings, which are incorporated into and constitute a part of this specification, illustrate one or more embodiments and, together with the detailed description, serve to explain the principles and implementations of the invention.

In the drawings:

FIGS. 1A and 1B illustrate an embodiment of the hemostatic pressure plug.

FIG. 2A and FIG. 2B illustrate the hemostatic pressure plug with a guidewire.

FIGS. 3A, 3B, and 3C illustrate the hemostatic pressure plug with an embodiment of a release mechanism.

FIGS. 4A, 4B, 4C, and 4D illustrate the hemostatic pressure plug positioned at a puncture site within the lumen of a blood vessel.

FIG. 5 is a side view of FIG. 4D illustrating the hemostatic pressure plug intervascularly positioned around an irregularly shaped blood vessel lumen.

FIGS. 6A, 6B, 6C, and 6D illustrate embodiments of release mechanisms.

FIGS. 7A, 7B, and 7C illustrate the hemostatic pressure plug used with an attachment mechanism.

FIGS. 8A, 8B, and 8C illustrate yet another embodiment of a release mechanism in accordance with an embodiment of the present invention.

FIGS. 9A and 9B illustrate yet another embodiment of a releasable mechanism used with a placement tube.

FIGS. 10A, 10B, and 10C illustrate still another embodiment of a releasable mechanism in an attached and detached mode.

FIG. 11 illustrates another embodiment of the hemostatic pressure device.

FIG. 12 illustrates a method for promoting hemostasis at a puncture site.

FIG. 13 illustrates another method for promoting hemostasis at a puncture site.

### DETAILED DESCRIPTION

Embodiments are described herein in the context of a hemostatic pressure plug. Those of ordinary skill in the art will realize that the following detailed description is illustrative only and is not intended to be in any way limiting. Other embodiments will readily suggest themselves to such skilled persons having the benefit of this disclosure. Reference will now be made in detail to implementations as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure

Providing hemostasis at a blood vessel puncture site is important for procedures such as percutaneous access to prevent bleeding and hematoma of a mammalian body or patient. Thus, a solution to facilitate hemostasis intervascularly at a puncture site may be achieved by deploying a flexible hemostatic plug within the blood vessel and utilizing the pressure difference between the inside and the outside of the blood vessel.

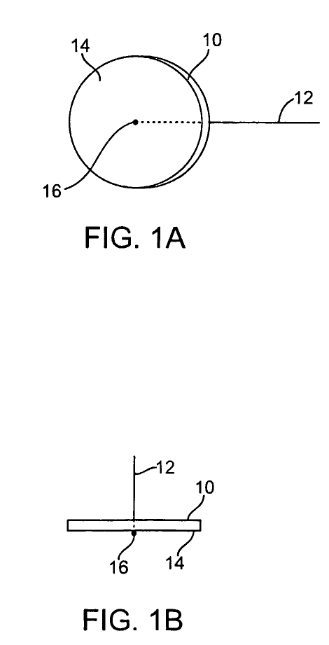

Referring now to FIGS. 1A and 1B, which illustrate an embodiment of the hemostatic pressure plug. FIG. 1A is a prospective view of the plug 10. The plug 10 is illustrated as being circular in shape, however, any shape may be used such as a square, oval, triangle, and any other shape. A release mechanism 12 may be releasably positioned near the center of the plug 10. FIG. 1A illustrates the release mechanism 12 as a thread, string, or suture. However, other release mechanisms, as further described in detail below, may be used. As illustrated in FIG. 1B, a side view of FIG. 1A, the thread or string 12 may be threaded through the plug 10 and held in position at the plug bottom 14 with a knot 16 at one end of the thread 12. However, the thread may be held in position within the plug by other means such as with the use of any adhesives or biocompatible polymers such as PGA, gelatin, mannitol and the like. Once the plug 10 is positioned at the puncture site as described in detail below, the thread 12 may be cut below the patient's skin line by depressing the patient's skin and cutting the thread 12.

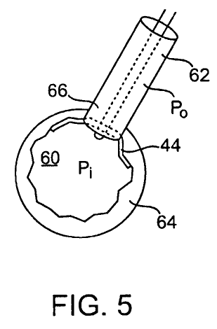

The plug 10 may have any diameter necessary to facilitate hemostasis at a puncture site. By way of example only and not intended to be limiting, a plug having a diameter of 3 mm to 6 mm may plug a blood vessel puncture having a diameter of 2.0 mm. The plug may also be formed with radial slits or cuts throughout the plug to provide for a more secure seal within an irregular blood vessel lumen (FIG. 5). The slits may be positioned about every 45° apart. The thickness of the plug may vary between 0.2 mm to 1.0 mm. The thinner the plug, the easier it is to deploy compared to thicker plugs. Further, if the plug is too thick or rigid, it may not be flexible enough to circumferentially cover and seal the puncture thereby resulting in blood oozing out of the puncture.

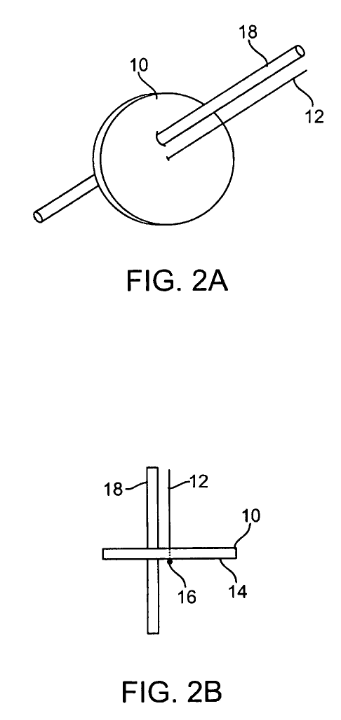

FIGS. 2A is a prospective view and FIG. 2B is a side view illustrating the hemostatic pressure plug with a guidewire. A guidewire 18 may be inserted at any position within the plug 10, however, it is advantageous to locate the guidewire 18 near the center of the plug 10 to provide for easier deployment and positioning of the plug 10.

When the guidewire 18 is removed from the plug 10, a hole will be formed in the plug 10 through which blood may flow through. However, the plug 10 may be made of any self-sealing biocompatible material as further described below. Thus, the hole may self-seal itself closed to prevent any flow of blood through the hole. Additionally, the guidewire hole may be surrounded by an expandable hemostatic material, such as foam and other materials as further discussed below, such that when the guidewire is removed from the hole, blood will cause the hemostatic material to expand and swell to seal the hole.

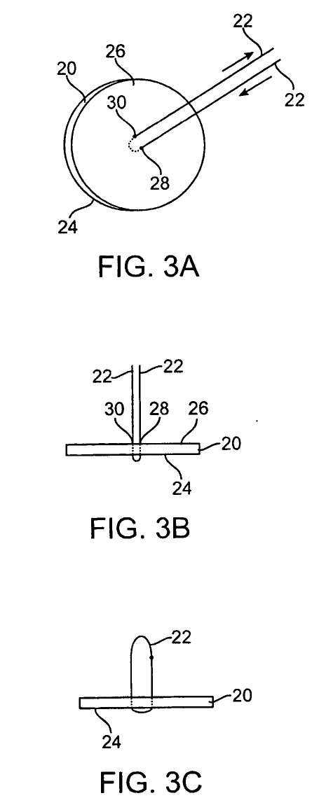

FIGS. 3A, 3B, and 3C illustrate the hemostatic pressure plug with an embodiment of a release mechanism. FIG. 3A is a prospective view of the plug 20 having a release mechanism 22 looped through the plug 20. The release mechanism 22 may be a thread or string as illustrated in FIGS. 1A and 1B above. However, in contrast to FIGS. 1A and 1B, the release mechanism is not tied in a knot at the plug bottom 24. Rather, the string is inserted through a first opening 28 from the plug top 26 through the plug bottom 24. The string is then inserted through a second opening 30 at the plug bottom 24 through the plug top 26 there by forming a loop through the plug 20. The first opening 28 and second opening 30 are positioned near the center of the plug 20. Thus, when the plug 20 is deployed and positioned at the puncture site as described in detail below, the thread 22 may be easily withdrawn from the patient by merely pulling or withdrawing one end of the thread 22. Alternatively, the release mechanism 22 may form a continuous loop through the plug by tying the ends of the string together as illustrated in FIG. 3C or both ends of the release mechanism 22 may be attached to the plug bottom 24 with knots (not shown) or any other means as described above.

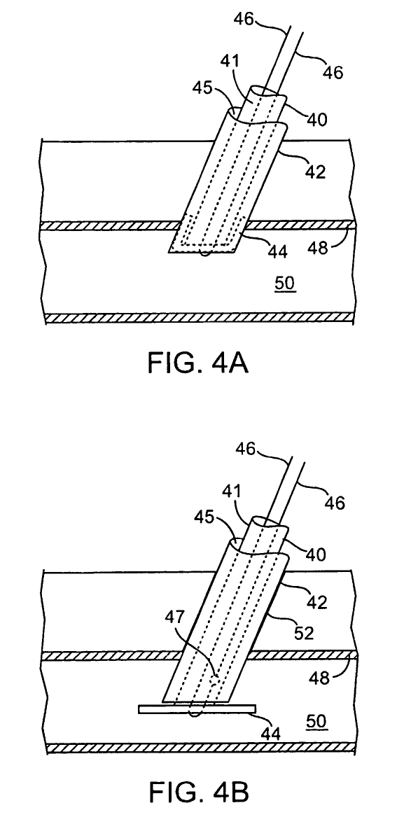

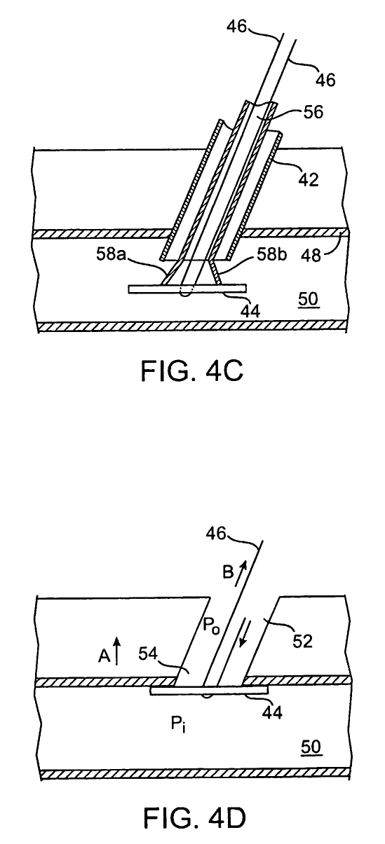

FIGS. 4A, 4B, 4C, and 4D illustrate the hemostatic pressure plug positioned at a puncture site within the lumen of a blood vessel. There are many methods known to those of ordinary skill in the art to deploy the plug at the puncture site. Thus, not every method will be discussed herein so as to not overcomplicate the present disclosure. However, a brief description of a few methods will be provided herein for illustratory purposes only and are not meant or intended to be limiting in any way.

FIG. 4A illustrates the plug 44 positioned within a first hollow tube 42, such as a sheath or an introducer. A second hollow tube 40, such as a pusher, may be positioned around the center of the plug 44 whereby the plug 44 surrounds one end of the pusher 40 and the release mechanism 46 may be received within the a lumen 41 of the pusher 40. The pusher 40 and plug 44 may then be inserted into the lumen 45 of the sheath 42. Although FIG. 4A is illustrated with the use of a sheath, the plug 44 may also be inserted into the tissue tract without the use of a sheath.

As illustrated in FIGS. 4B and 4C the plug 44 and release mechanism 46 are inserted into the sheath 42 and simultaneously pushed toward the blood vessel 48 with the pusher 40, 56. As illustrated in FIG. 4B, the pusher 40 or the sheath 42 may have an entrance port 47 for bleed back indication to locate the blood vessel puncture site, as further described below. As illustrated in FIG. 4C, the pusher 56 may be a second deployment device having expandable members 58a, 58b at the pusher bottom. The expandable members 58a, 58b assist to expand the plug 44 intervascularly or within the blood vessel lumen 50. This prevents the plug 44 from folding onto itself.

Referring to FIG. 4D, once the plug is exposed within the blood vessel lumen 50, the pusher 40, 56 and sheath 42 may be removed from the tissue tract 52. The plug 44 may be pulled closer to the puncture 54 by pulling both ends of the release mechanism 46 away from the blood vessel or patients skin. However, only a slight tug or pull is necessary. The pressure Pᵢ within the blood vessel lumen 50 is greater than the pressure Pₒ within the tissue tract 52. This pressure difference causes the plug 44 to be sucked into the puncture in the direction of arrow A thereby surrounding the puncture 54 and blocking blood flow out of the puncture 54. It is also this pressure difference which allows the plug 44 to be securely positioned around the puncture 54. A user may know when the plug 44 is positioned around the puncture through visual indication, such as lack of bleeding out of the tissue tract or a bleed back indicator as discussed below, or tactile feel, such as when the user feels an increase in tension when pulling on the release mechanism. When visual indication is used to determine whether the plug is secured around the puncture site, it is preferable that a large amount of bleed back outflow be observed, such as greater than 1 cc/sec of outflow. Bleed back, as further described in detail below may be observed out of the sheath, pusher, or tissue tract. Once positioned around the puncture 54, the release mechanism may be withdrawn out of the patient by withdrawing one end of the thread in the direction of arrow B.

FIG. 5 is a side view of FIG. 4D illustrating the plug intervascularly positioned around an irregularly shaped blood vessel lumen. As described above, the pressure inside Pᵢ the blood vessel lumen 60 is greater than the pressure outside Pₒ the blood vessel (i.e. such as the tissue tract 62). This pressure difference, the flexibility of the plug 44, and its circumferential coverage and extension over the puncture 66 securely positions the plug 44 against the blood vessel wall 64 and around the puncture 66, even if the blood vessel wall 64 is irregular in shape. This is important to provide a tight and secure seal around the puncture 66 to prevent blood from oozing out of the blood vessel 60. Current devices with rigid anchors, especially those which do not provide circumferential coverage around the puncture site are prone to blood leaking or oozing out of the blood vessel.

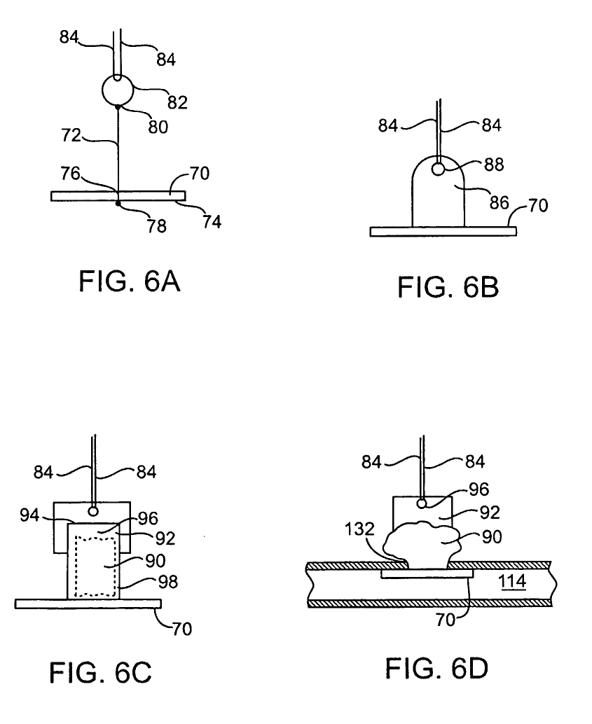

FIGS. 6A, 6B, 6C, and 6D illustrate embodiments of release mechanisms. FIG. 6A illustrates the plug 70 utilizing the same release mechanism described in FIGS. 3A and 3B. A thread or string 72 may be positioned near the center the of plug 70. A first end 76 of the thread may be attached to the plug bottom 74 with a knot 78 or any other secure means. The second end 80 of the thread 72 may be attached to an O-ring 82. The release mechanism 84 may be looped through the o-ring 82 whereby once the plug 70 is positioned around the puncture, the release mechanism 84 may be withdrawn from the patient as described above with reference to FIGS. 3A, 3B, and 4D. In this embodiment, it is preferable that the thread 72 and o-ring 82 be made of any absorbable, biocompatible material as further described below. Additionally, FIG. 6A is illustrated using an o-ring, however, the o-ring is not intended to be limiting as any other device may be used. For example, as illustrated in FIG. 6B, the plug 70 may be formed with a resilient extension member 86 having an opening 88. The release mechanism 84 may be looped through the opening 88. Alternatively, the release mechanism may be secured to extension member 86 by tying one end of the release mechanism 84 to itself after being looped through opening 88.

FIG. 6C illustrates the use of a hemostatic material removably attached to the plug. The hemostatic material 90 may be removably attached near the center of the plug 70 with the use of any biocompatible polymers such as PGA, gelatin, mannitol and the like. Alternatively, the hemostatic material 90 may be incorporated into the plug 70. The hemostatic material 90 may be a gelatin sponge or collagen which may further be contained in a gelatin capsule 98 as described below. In another embodiment, the extension member 92 may be surrounded with hemostatic material (not shown) which in turn may be contained in a gelatin capsule 98. As illustrated in FIG. 6D, when the plug 70 is positioned around the puncture 132 and the capsule is exposed to blood or other fluids, the capsule will dissolve thereby releasing the hemostatic material 90. The hemostatic material may then absorb the fluids and expand to provide hemostasis at the puncture site 132.

The capsule 98 may be advantageously made from gelatin and formulated to have flexibility (like a gel-cap vitamin E) or be stiff like a typical 2-piece oral capsule. Capsules are made to dissolve within a predetermined time, with a dissolution time between 10 seconds and 10 days, and normally between one minute and 10 minutes. Also, the capsule 98 can be formulated to be inert (e.g. non thrombogenic, non-bacteriostatic) or to provide/deliver therapeutic benefit (e.g. bacteriostatic, clot acceleration which may include clot accelerators such as thrombin, calcium based compounds, chitosan, and may also include antibiotics or radiopaque substances). The capsule 98 can vary in characteristics along its length. For example, the distal region can be inert while the proximal region comprises therapeutic material.

The release mechanism 84 may be looped through the capsule 98 or looped through an extension member 92, having an opening 96, attached to the capsule top 94. The capsule 90 may plug the puncture to ensure that blood will not flow out the blood vessel 14 and may swell slightly to securely control the puncture.

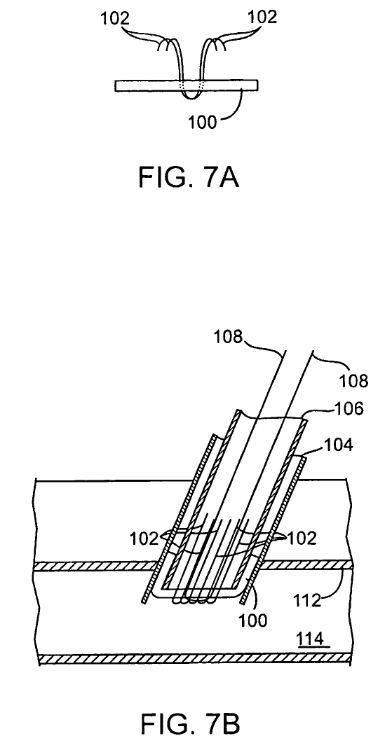

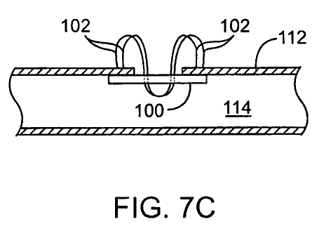

FIGS. 7A, 7B, and 7C illustrate the hemostatic pressure plug used with an attachment mechanism. Referring to FIG. 7A, the plug 100 may be used with an attachment mechanism 102 looped near the center of the plug 100 illustrated without a release mechanism for clarity. However, any type of release mechanism may be used with the attachment mechanism 102. The attachment mechanism 102 may be a plurality of hooks that are compressed when enclosed within the lumen of a tube and expand when exposed. The hooks grasp the outside of the blood vessel and/or the tissue tract to secure the plug 100 to the puncture. The hooks may be flexible to prevent puncturing the blood vessel wall 112 or the hooks may be strong enough to puncture and attach into the blood vessel wall 112. As illustrated in FIG. 7B, the plug 100 may be pushed through the sheath 104 with a pusher 106. The release mechanism 108 and hooks 102 may be positioned within the pusher 106. Once the plug 100 is positioned at the puncture site 110, as illustrated in FIG. 7C, the pusher may be withdrawn thereby exposing the hooks 102, which expand and grasp the outside of the blood vessel 112. The attachment mechanism 102 ensures that the plug 100 will remain in position within the blood vessel lumen 114. The description of the attachment mechanism as releasable hooks is not intended to be limiting. Other attachment mechanisms maybe utilized to secure the plug to the blood vessel such as barbs, and the like.

The attachment mechanism may be encased with an expandable hemostatic material, such as a sponge or foam and other materials as further discussed below. When the hooks are released, the expandable hemostatic material may swell and expand to seal any holes which may be formed from the hooks as well as the puncture and adjacent tissue tract. This will further provide another mechanism to securely block blood flow out of the blood vessel.

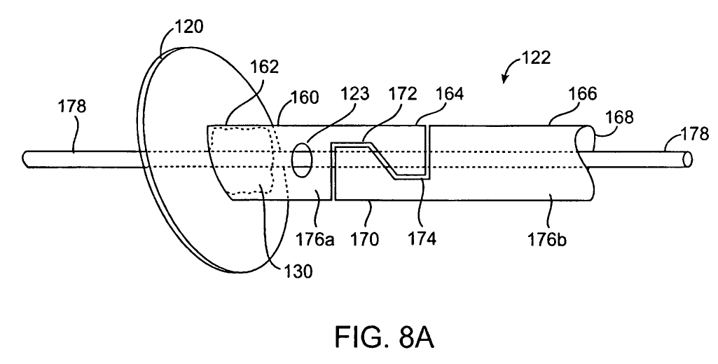

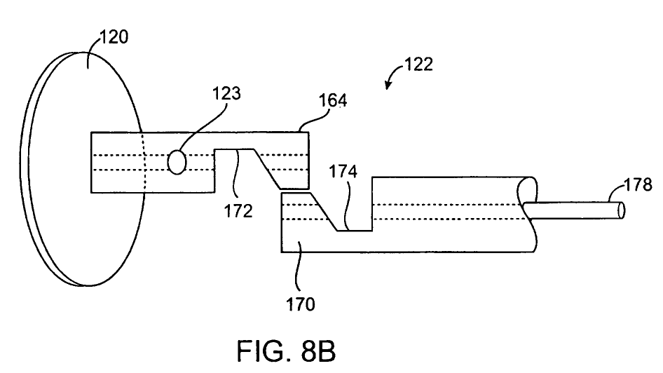

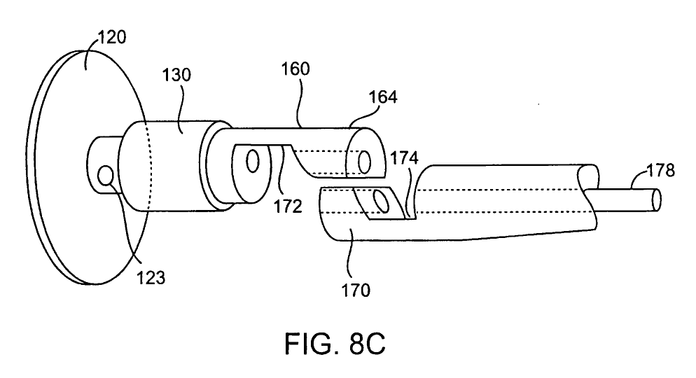

FIGS. 8A, 8B, and 8C illustrate yet another embodiment of a release mechanism. As shown in FIG. 8A, the plug 120 may have a releasable mechanism, generally numbered as 122, near the center of the plug 120. The release mechanism 122, may have an entrance port 123 for bleed back indication to locate the blood vessel puncture site, as further described below. The releasable mechanism 122 has a first connector 160 having a first end 162 and a second end 164 and a second connector 166 having a top 168 and a bottom 170. The first connector 160 has a first notch 172 at the second end 164 to releasably mate with the second connector bottom 170. The first connector 160 may be attached near the center of the plug 120. The second connector 166 has a second notch 174 at the bottom 170 to releasably mate with the first connector second end 164. The first connector 160 and second connector 166 may have a lumen 176a and 176b to receive a guidewire 178 or any other device. Once the first connector 160 and the second connector 166 are mated at the first notch 172 and second notch 174, the guidewire 178 may be placed through the releasable mechanism lumen 176a and 176b. The guidewire 178 may assist in preventing the first connector 160 and the second connector 166 from separating but will also allow the releasable mechanism to move axially along the length of the guidewire 178. Although FIG. 8A is illustrated with the use of a guidewire, the release mechanism 122 may be used without a lumen 176a, 176b and guidewire 178 and may be engaged with other devices such as a pusher or sheath, and released when the device is withdrawn.

FIGS. 8B and 8C illustrate the releasable mechanism of FIG. 8A in a detached mode. Once the plug 120 is positioned at the puncture site, the guidewire 178 is withdrawn and the releasable mechanism may be detached by detaching the second connector bottom 170 from the first notch 172 and the first connector top 164 from the second notch 174. The releasable mechanism may be detached by a gentle pull or by twisting the releasable mechanism such that the second connector bottom 170 is positioned opposite the first notch 172 and the first connector top 164 is positioned opposite the second notch 174. The method of detaching the releasable mechanism 122 is not meant to be limiting as there may be different ways to release the mechanism. However, this provides a low-force, stable way to release the plug 120 at the blood vessel puncture site and withdraw any devices used such as the guidewire 178.

Alternatively, as illustrated in FIG. 8C, a hemostatic material 130 may be positioned around the first connector 160 above the entrance port 123. The hemostatic pressure plug 120 may be delivered through a tissue tract with the use of a sheath already in the lumen until the entrance port 123 and plug 120 are exposed through the blood vessel lumen. Blood entering the entrance port 123 will travel through lumens 176a, 176b and out an exit port (not shown) such that bleed back may be observed by a user which is an indication that the plug 120 is within the blood vessel lumen. The user may then withdrawn the plug 120 with the use of the release mechanism until the bleed back indication ceases, which is an indication of the location of the blood vessel puncture.

When the guidewire 178 is removed from the plug 120, a hole will be formed in the plug 120 that will allow blood to flow through. However, the plug 120 may be made of any self-sealing absorbable material as further described below. Thus, the hole may self-seal itself closed to prevent any flow of blood through the hole. Additionally, the guidewire hole may be made of an expandable hemostatic material, such as foam and other materials as further discussed below, such that when the guidewire 178 is removed from the hole, the expandable hemostatic material may swell and expand to seal the hole. Alternatively, as illustrated in FIGS. 8A and 8C, the hemostatic material 130 may be positioned within lumen 176a or surrounding a portion of first connector 160. When the guidewire 178 is removed and blood enters the lumen 176a, the hemostatic material will swell and expand to seal the hole and puncture.

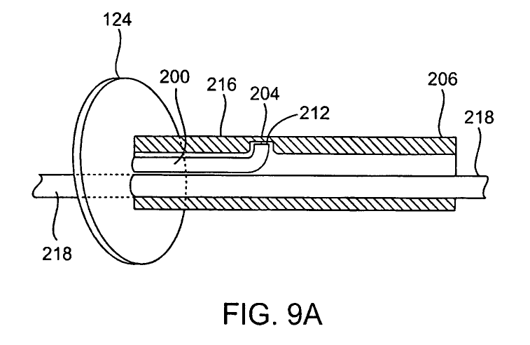

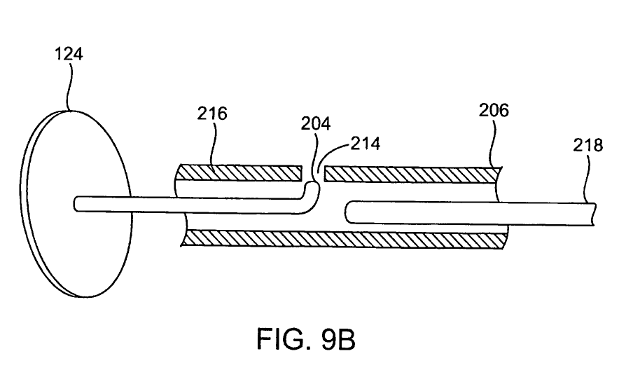

FIGS. 9A and 9B illustrate yet another embodiment of a releasable mechanism used with a placement tube. FIG. 9A illustrates the plug 124 having a release mechanism 200 with a foot 204 at one end. The release mechanism 200 may be releasably attached to the center of the plug 124. The release mechanism 200 may be used with a placement tube 206 having a recess 212 in its wall to mate with a foot 204. The recess 212 may extend partially into the wall of the placement tube 206 as shown in FIG. 9A or the recess 214 may extend through the entire wall of the placement tube 206 as shown in FIG. 9B. The recess, 212 or 214, is preferably located near the placement tube bottom 216, but may be positioned at any location along the placement tube 206.

As shown in FIG. 9A, the foot 204 is held and engaged within the recess 212 by a guidewire 218. Once the plug 124 is positioned at the puncture site, the release mechanism may be released by removing the guidewire 218 as shown in FIG. 9B. Removing the guidewire 218 will cause the foot 204 to disengage from the recess 214. This provides for an efficient and simple release mechanism to release the plug 124 without any tugging or pulling that may reposition the plug or cause damage to the surrounding tissue or puncture site.

When the guidewire 218 is removed from the plug 124 a hole will be formed in the plug 124 that will allow blood to flow through. However, the plug 124 may be made of any self-sealing absorbable material as further described below. Thus, the hole may self-seal itself closed to prevent any flow of blood through the hold. Additionally, the guidewire hole may be made of an expandable hemostatic material, such as foam and other materials as further discussed below, such that when the guidewire 218 is removed from the hole, the expandable hemostatic material may swell and expand to seal the hole.

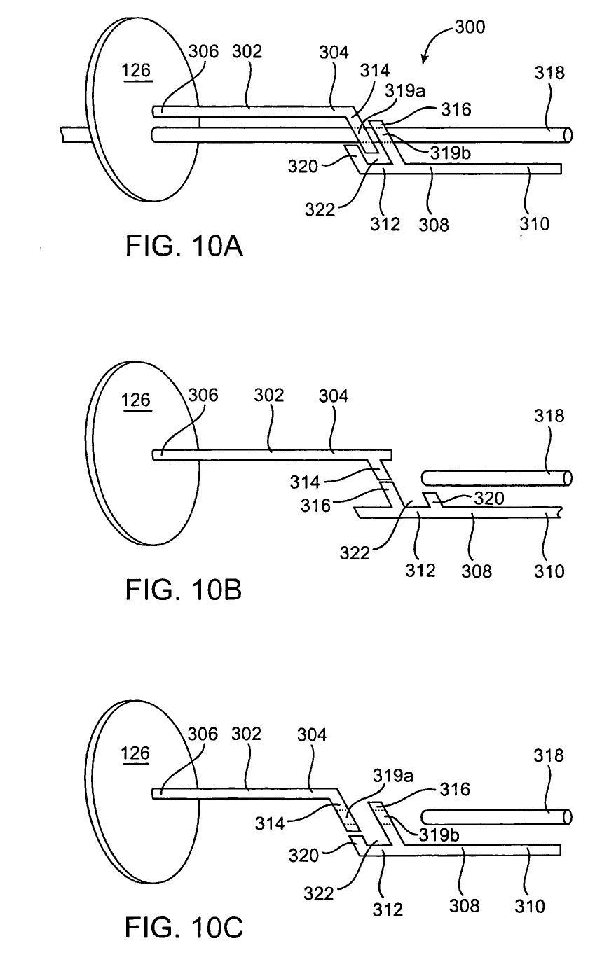

FIGS. 10A, 10B, and 10C illustrate still another embodiment of a releasable mechanism in an attached and detached mode, respectively. The releasable mechanism, generally numbered 300, has a first connector 302 having a first end 306 and a second end 304 and a second connector 308 having a top 310 and a bottom 312. The first connector first end 306 may be attached near the center of the plug 126. The second connector top 310 may extend beyond a patient's skin to allow a user to release the release mechanism from the plug 126.

The first connector second end 304 has a first ring 314 positioned at an angle away from the second end 304. The second connector 308 has a projection 320 parallel to a second ring 316 near the bottom 312 such that the projection 320 and the second ring 316 form a recess 322 to releasably mate with the first ring 314. The projection 320 may be shorter in length that the second ring 316. Both the first ring 314 and the second ring 316 have a lumen 319a, 319b to receive a guidewire 318.

As shown in FIG. 10B, the location of the first ring 314, second ring 316, and projection 320 are not meant to be limiting. For example, the projection 320 may be in front of the second ring 316 as shown in FIG. 10B or may be behind the second ring 316 as shown in FIG. 10C. Additionally, the first ring 314 may be located at the second end 304 as illustrated in FIG. 10C or may be located near the second end 304 as illustrated in FIG. 10B. Thus, it may be appreciated that there are many different placements for the first ring, second ring, and projection.

In use, the first ring 314 is positioned within the recess 322 and the guidewire 318 is positioned through lumens 319a, 319b. The guidewire 318 will assist in preventing the first connector 302 and the second connector 308 from separating but will allow the releasable mechanism to move axially along the length of the guidewire 318. Once the plug 126 is positioned at the puncture site, the guidewire 318 is removed and the first ring 314 may be released from the recess 322 with a gentle tug or twist such that the first ring 314 is no longer within the recess 322 as shown in FIGS. 10B and 10C.

When the guidewire 318 is removed from the plug 126 a hole will be formed in the plug 126 that will allow blood to flow through. However, the plug 126 may be made of any self-sealing absorbable material as further described below. Thus, the hole may self-seal itself closed to prevent any flow of blood through the hold. Additionally, the guidewire hole may be made of an expandable hemostatic material, such as foam and other materials as further discussed below, such that when the guidewire 318 is removed from the hole, the expandable hemostatic material may swell and expand to seal the hole.

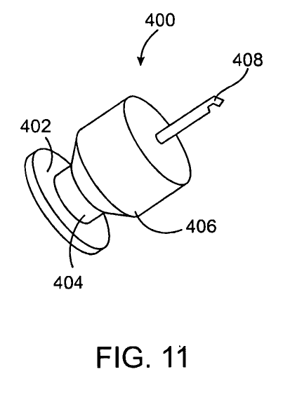

FIG. 11 illustrates another embodiment of the hemostatic pressure device. The device, generally numbered 400, comprises a disk 402 attached to a neck 404 which is attached to a body 406. In use, the device 400 would be compressed radially for placement through the tissue tract with the use of a sheath, pusher, or release mechanism.

The disk 402 may be similar to the hemostatic pressure plug described above. The disk will circumferentially intervascularly seal and cover the puncture site. The device 400 may have a release mechanism 408 attached near the center of the body 406 opposite from the neck 404. Since several possible embodiments of the release mechanism are discussed in detail above, it will not be discussed further herein.

Neck 404 may by attached near the center of disk 402 at one side. In use, neck 404 will be positioned within the blood vessel wall. Thus, neck 402 may have a smaller diameter than the disk 402 and body 406 such that when neck 402 is positioned within the blood vessel puncture wall, it will not tear or rip the blood vessel wall. Body 406 may be attached to neck 402 opposite the side where neck 404 is attached to the disk 402. Body 406 may be any hemostatic material such as the hemostatic material detailed above. Body 406 may expand to provide additional intravascular sealing of the blood vessel puncture.

Although disk 402, neck 404, and body 406 may be made of the same materials as discussed in detail below, it is preferable that disk 402 has enhanced properties of density, strength, and resilience. The enhanced properties of disk 402 may be achieved through heat setting and pressure to permanently set the disk axially as a more dense, thinner form. By way of example only, heat from about 200° F. to 400° F. and pressure from as little as 15 psi may be used to set the disk. The neck may also be modified, for instance by radial heat setting, to a more dense, smaller diameter all the while maintaining at least some of its ability to expand upon exposure to blood or fluids.

The device 400 may be selectively coated with known substances to slow their expansion and/or absorption rates. The device 400 may also be coated with absorbable or non-absorbable polymers and dispersions and soaked or wicked with any desired absorbable or non-absorbable polymers and dispersions for delivery to the blood vessel puncture site.

The various releasable mechanisms described above are illustrated as cylindrical or rod shaped. However, the releasable mechanisms may be any shape such as a rod, square, or other shape. Additionally, the embodiments described above were illustrated with reference to a releasable mechanism and plug used with a guidewire. However, there are other applications the releasable mechanism may be used with such as neurological surgery devices and coils.

The plug may be made of any semi-rigid, absorbable, biocompatible material such as Collagen, Oxidized Cellulose, PGA, methyl cellulose, carboxymethyl cellulose, carbowaxes, gelatin (particularly pigskin gelatin), urethane foam, and sugar based compounds. Among the other suitable polymers are polylactic glycolic acids, polyvinyl pyrrolidone, polyvinyl alcohol, polyproline, and polyethylene oxide. Alternatively, the plug may be made of a non-absorbable material such as dacron, gortex, felt, suede, urethane foam, and any other cross-linked or fixed xenograft materials. The plug should not be made of a flimsy material that does not retain its shape because it will be difficult to position the plug at the puncture site and the plug will not be able to securely block the entire puncture. The plug requires some memory such that it can substantially retain its original shape after being compressed or folded when delivered through the tissue tract, sheath, or any other delivery device. The plug should not be made of a rigid material or it will not conform to the shape of or be pressure sealed to the puncture thereby resulting in the oozing of blood out of the blood vessel puncture.

The release mechanisms, guidewire, attachment mechanism, and hemostatic material described above may be made of any type of absorbable, biocompatible material as described above. The hemostatic material may also be made of other materials such as fibrillar collagen, collagen sponge, regenerated oxidized cellulose, gelatin powder, hydrogel particles. Alternatively, the release mechanisms, guidewire, and attachment mechanism may be made of a non-absorbable material such as any biocompatible textile material, non-absorbable plastics, Nitinol, stainless steel, and the like.

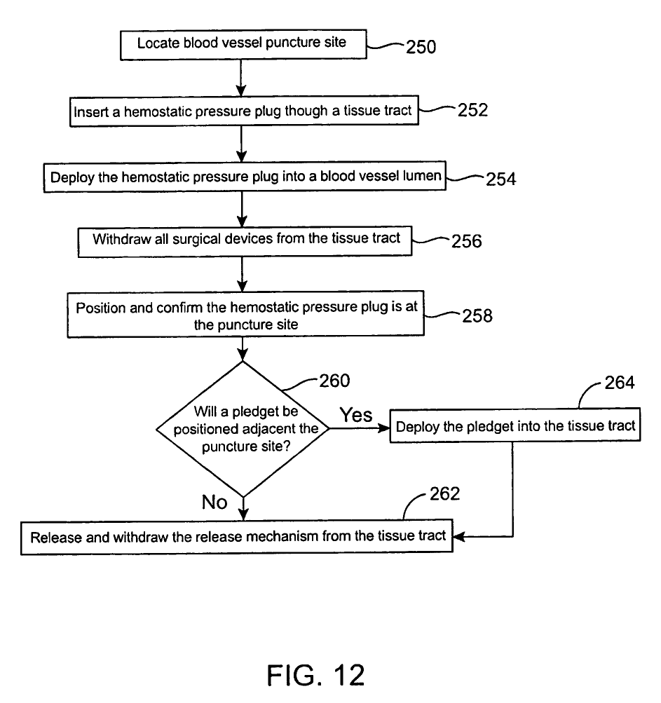

FIG. 12 illustrates a method for promoting hemostasis at a puncture site. After a surgical procedure is complete, the puncture site must be sealed to control bleeding from the punctured artery. The blood vessel puncture is located at 250. There are various methods to locate the blood vessel puncture site, of which any of the methods may be used with the embodiments described above. By way of example only, and not intended to be limiting, a depth indicator or marker on the sheath, pusher, or introducer may be used to locate the blood vessel puncture. Other methods, such as the use of a bleed back indicator illustrated on the pusher in FIG. 4B or on the release mechanism in FIG. 8C, may be used to locate the puncture site. The various methods which may be used to locate the puncture site will not be described herein so as to not overcomplicate the present disclosure.

Once the blood vessel puncture site is located, the hemostatic pressure plug is inserted into the tissue tract at 252. The plug may be inserted into the tissue tract by any means, such as with the use of a sheath and pusher or with any of the release mechanisms described above. The hemostatic pressure plug is pushed into the tissue tract until it is deployed into the blood vessel lumen at 254. All surgical devices are withdrawn from the tissue tract at 256 and the plug is positioned and confirmed that it is at the puncture site at 258.

The plug may be positioned at the puncture site with only a slight pull of the release mechanism in a direction away from the blood vessel or away from the patient's skin. The pressure within the blood vessel lumen is greater than the pressure within the tissue tract. This pressure difference causes the plug to be sucked into and around the puncture thereby surrounding the puncture and blocking blood flow out of the puncture. It is also this pressure difference which allows the plug to be securely positioned around the puncture. Confirmation that the plug is located at the blood vessel puncture site may be completed through visual indication, such as lack of bleeding out of the tissue tract or out of a bleed back indicator as discussed below, or tactile feel, such as when the user feels an increase in tension when pulling on the release mechanism.

Once the plug is securely positioned around the puncture, a pledget or hemostasis material may be deployed adjacent the puncture site at 260. The hemostasis material may be delivered to the puncture through the tissue tract 264 by any means and will not be discussed herein to prevent obfuscation of the present disclosure. However, by way of example only and not intended to be limiting, the pledget may be inserted through the release mechanism or by fluid pressure with the use of a sheath. If a pledget is not utilized, the release mechanism may be released and withdrawn from the tissue tract at 262.

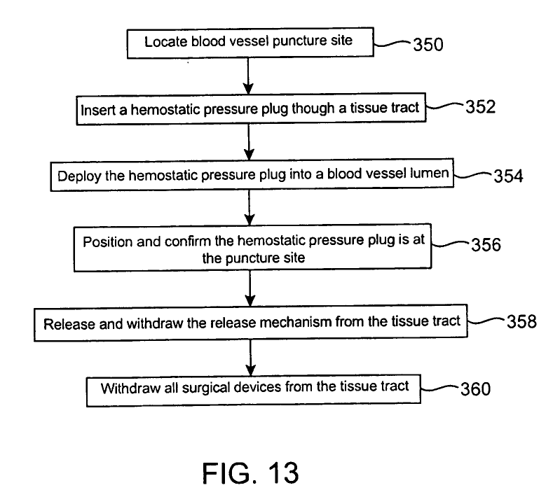

FIG. 13 illustrates another method for promoting hemostasis at a puncture site. The blood vessel puncture may be located at 350 through any method described above. Once the puncture site is located, the hemostatic pressure plug is inserted into the tissue tract at 352. The hemostatic pressure plug may then be pushed into the tissue tract until it is deployed into the blood vessel lumen at 354. The plug may be positioned and confirmed that it is at the puncture site at 356.

The plug may be positioned at the puncture site with only a slight pull of the release mechanism in a direction away from the blood vessel or away from the patient's skin. The pressure within the blood vessel lumen is greater than the pressure within the tissue tract. This pressure difference causes the plug to be sucked into and around the puncture thereby surrounding the puncture and blocking blood flow out of the puncture. It is also this pressure difference which allows the plug to be securely positioned around the puncture. Confirmation that the plug is located at the blood vessel puncture site may be completed through visual indication, such as lack of bleeding out of the tissue tract or out of a bleed back indicator as discussed below, or tactile feel, such as when the user feels an increase in tension when pulling on the release mechanism.

Once the plug is securely positioned around the puncture, the release mechanism may be released and withdrawn from the tissue tract at 358. All surgical devices may then be withdrawn from the tissue tract at 360.

While embodiments and applications have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

1. An apparatus to intervascularly promote hemostasis at a blood vessel puncture site having an inner lumen pressure and an outer lumen pressure, comprising:
a flexible plug having a center, a top surface, and a bottom surface; and
a release mechanism including a hemostatic material coupled to the center of the flexible plug and a resilient extension member coupled to the hemostatic material opposite the flexible plug, the release mechanism positioning and releasing the flexible plug intervascularly at the blood vessel puncture site;
wherein the inner lumen pressure is greater than the outer lumen pressure.

2-26. (canceled)

27. The apparatus of claim 1 wherein the hemostatic material is encapsulated in a biocompatible dissolvable capsule.

28. The apparatus of claim 27 further comprising a suture looped through the aperture.

29. An apparatus to position and release a flexible plug at a blood vessel puncture site, comprising:
a first connector having a lumen, a first end, second end, a first notch positioned neat the second end, said first connector first end coupled to a center of the flexible plug;
a second connector having a lumen, a top, a bottom, and a second notch positioned near the bottom;
wherein the second connector bottom is received by the first notch and the first connector second end is received by the second notch.

30. The apparatus of claim 29 further comprising a guidewire received by the second connector lumen and the first connector lumen to secure the first connector and the second connector together.

31. The apparatus of claim 29 wherein the first connector further comprises an entrance port positioned substantially near the first end to receive a flow of blood from the blood vessel.

32. The apparatus of claim 31 wherein the second connector further comprises an exit port positioned substantially near the second end top, wherein the flow of blood entering the entrance port exits.

33. The apparatus of claim 29 further comprising a hemostatic material coupled to the first connector first end.

34-39. (canceled)

40. An apparatus to promote hemostasis at a blood vessel puncture site having an inner lumen pressure and an outer lumen pressure, comprising:
a flexible disk to intervascularly seal a blood vessel puncture site;
a hemostatic body to intravascularly seal the blood vessel puncture site; and
a connector to couple the flexible disk to the hemostatic body, the connector positioned within a wall of the blood vessel puncture site;
wherein the inner lumen pressure is greater than the outer lumen pressure to forceably secure said flexible disk around the blood vessel puncture site.

41. The apparatus of claim 40 wherein the connector has a smaller diameter than a flexible disk diameter and a hemostatic body diameter.

42. The apparatus of claim 40 further comprising a release mechanism coupled to the hemostatic body.

43. The apparatus of claim 42 wherein the release mechanism is a suture having a first end secured with an adhesive to the hemostatic body.

44. The apparatus of claim 40 wherein the release mechanism comprises a resilient extension member coupled to the center of the hemostatic body, the resilient extension member having an aperture at a top.

45. The apparatus of claim 44 further comprising a suture looped through the aperture.

46. The apparatus of claim 44 wherein the resilient extension member is made of hemostatic material.

47. The apparatus of claim 46 wherein the extension member is encapsulated with a biocompatible dissolvable capsule.

48. The apparatus of claim 44 wherein the resilient extension member further comprises a hemostatic material positioned at a center of the resilient extension member.

49. The apparatus of claim 48 wherein the resilient extension member is encapsulated with a biocompatible dissolvable capsule.

50-58. (canceled)
